(19) United States
(12) Patent Application Publication
(10) Pub. No.: US 2017/0007472 A1
(43) Pub. Date: Jan. 12, 2017

(54) Pamper With Built in Disposable Scent Wrap/Bag

(71) Applicants: Damien Terrell Ambrose, Indianapolis, IN (US); Saraya R. Scott, Indianapolis, IN (US)

(72) Inventors: Damien Terrell Ambrose, Indianapolis, IN (US); Saraya R. Scott, Indianapolis, IN (US)

(21) Appl. No.: 14/996,199

(22) Filed: Jan. 14, 2016

Related U.S. Application Data
(60) Provisional application No. 62/191,346, filed on Jul. 11, 2015.

(51) Int. Cl. A61F 13/551; A61F 13/84; A61F 13/15; B65D 75/58; B65D 33/28

(52) U.S. Cl. CPC A61F 13/5512; B65D 75/5855; B65D 33/28; A61F 13/15756; A61F 13/8405; A61F 2013/55125; A61F 2013/8408

(57) ABSTRACT

Presently when a pamper/pullup is used and discarded it is gathered and wrapped into itself and disposed of with no odor protection which results in foul odors exuding from a pamper/pull up. This invention is designed with a pullback tab and concealed compartment on the upper rear that holds a scented disposable bag to eliminate smell, Therefore, allowing a fresh disposable wrap/bag for pamper changing convenience.

### CROSS REFERENCE TO RELATED APPLICATIONS

Not Applicable

### FEDERALLY SPONSORED RESEARCH

Not Applicable

### SEQUENCE LISTINGS OR PROGRAM

Not Applicable

### FIELD OF THE INVENTION

The present invention relates to pampers for babies or adults with a concealed scent wrap for odorless disposal.

### BACKGROUND OF THE INVENTION

Pampers/pull-ups, primarily worn by infants, children and adults, in certain circumstances allows one to defecate or urinate without the use of the toilet. When pampers becomes soiled it then requires changing. Pampers are usually changed by a parent or caretaker and is typically disposed of in a trash can without any odor protection. Thereby causing foul smell in the air and a longer foul odor if a pamper/pull up sits in the trash over time.

Although, there are other ways to eliminate the foul odor of a soiled pamper none have proven to eliminate odor. For example, placing soiled pamper directly in trash, placing pamper in a separate trash bag before disposing in trash or adding a fresh scent at the bottom of the trash can. These alternatives are not effective because a single trash liner does not effectively cover the odor of defecate and when changing in public it is not always guaranteed every trash can will have an added fresh scent to conceal foul smell. In situations when an emergency changing is needed there is no convenient way to dispose of pampers outside normal changing areas.

There is a published application from inventor Paul J. Rossi which was published May 30, 2002 and abandoned later by the Inventor. Application number U.S. Ser. No. 09/803,826 Publication number US20020065500 A1. Although the titles of the inventions are similar, the embodiments and specifications are diverse in various aspects.

### OBJECTS AND ADVANTAGES

Accordingly, besides the objects and advantages of the concealed disposable built in trash bag described in my above patent, several objects and advantages of the present invention are:
(a) to provide a pamper that can be produced with convenient and rapid and economical change in a pamper/pull ups that are being produced;
(b) to provide a disposable scented bag in each individual pamper;
(c) to provide a seal for scented disposable bag to stay fresh until ready for use;
(d) to provide a built-in scented disposable bag that will not be harmful or exposed while worn;
(e) to provide a convenient way to change pampers outside of normal changing areas.

Further objects and advantages are to provide a pullback tab and concealed compartment on the upper rear that holds a scented wrapper/bag that is used to eliminate smell and convenient disposal. This disposable method eliminates foul odor from exuding from a pamper/pull up. It is also available on each individual pamper for convenience. Therefore, it is more necessary to use this pamper for a more useful changing. Scented bag is already available so there is no need to purchase separate trash liners for disposal and changing can also be done in public without the embarrassment of the foul scent left in public restrooms. Still further objects and advantages will become apparent from a consideration of the ensuing description and drawings.

### BRIEF DESCRIPTION OF THE INVENTION

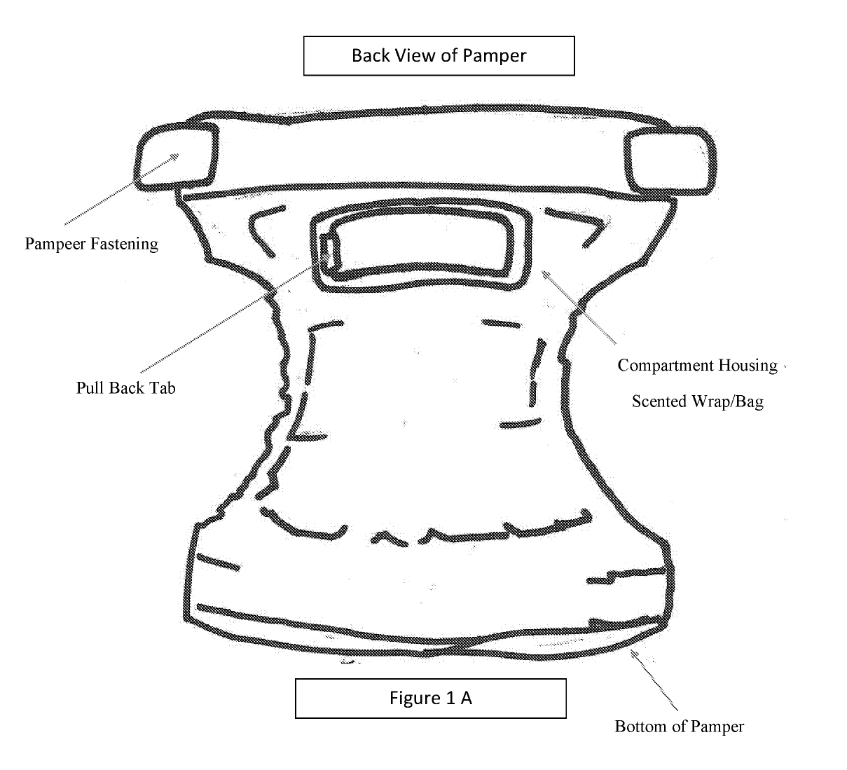

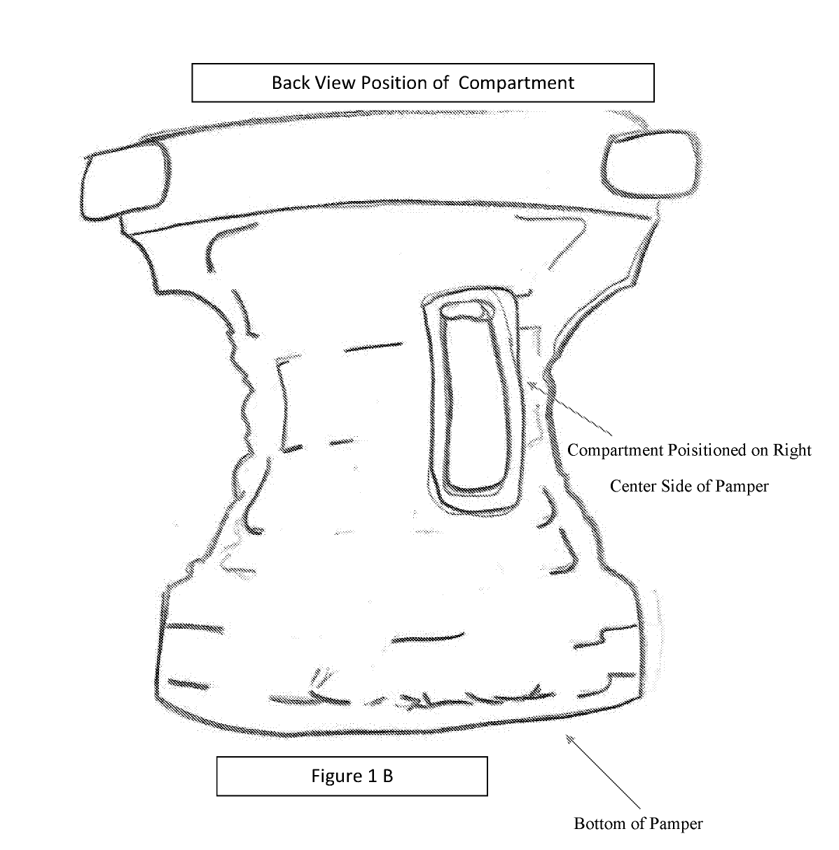

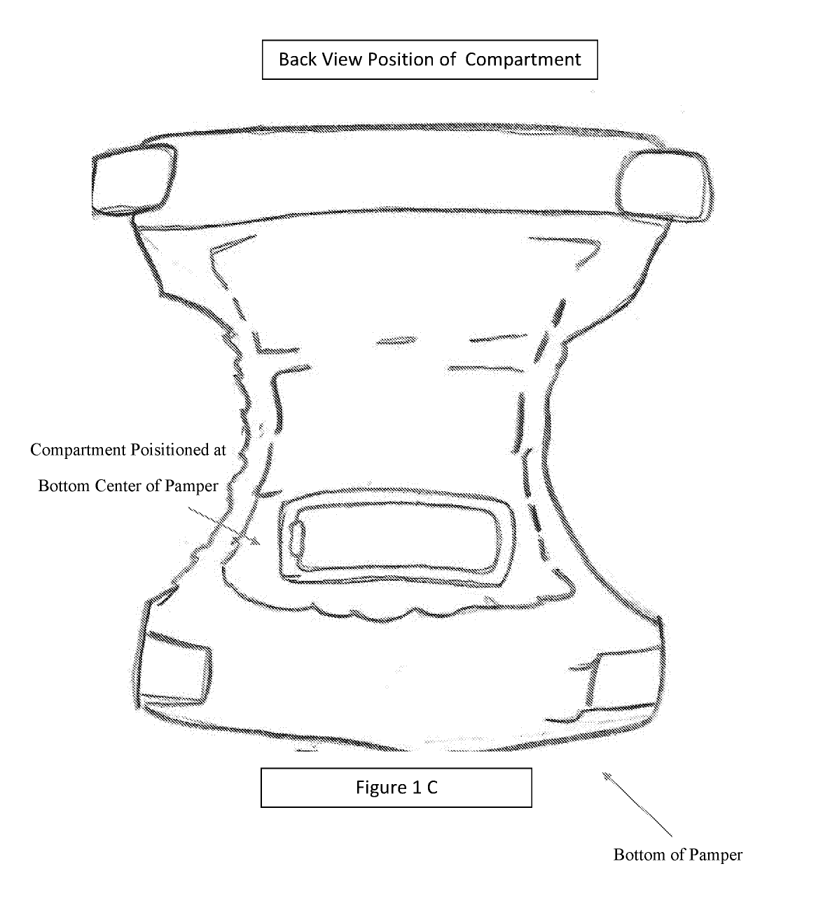

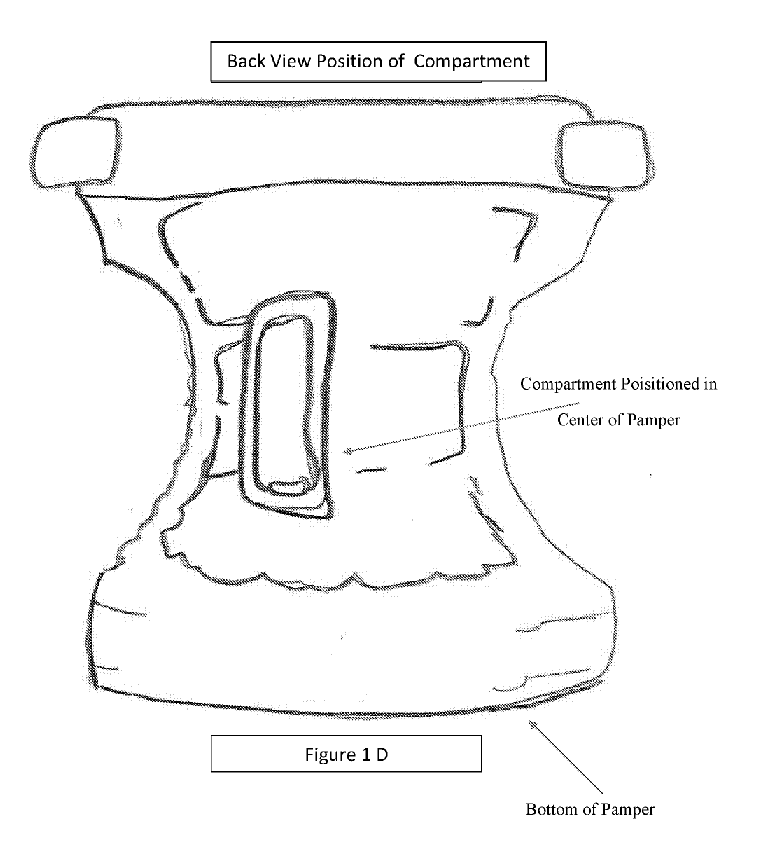

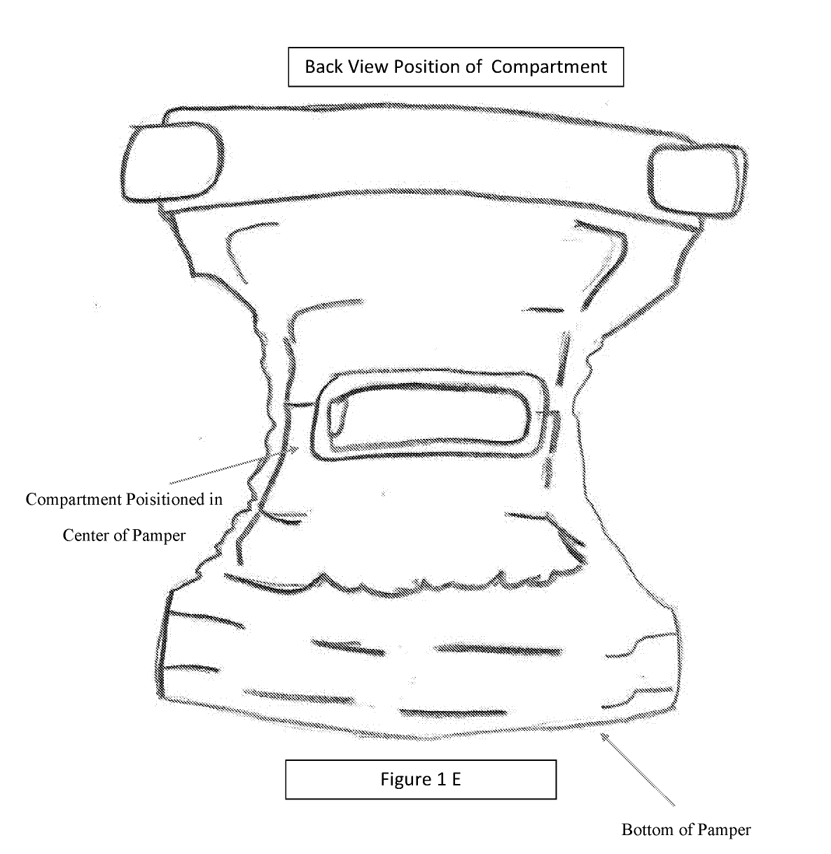

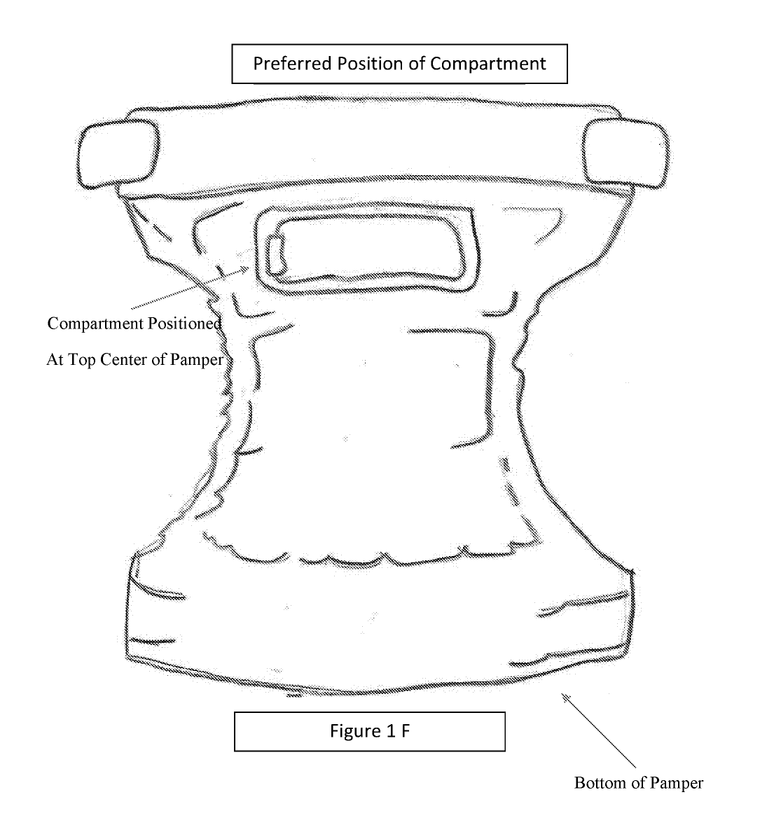

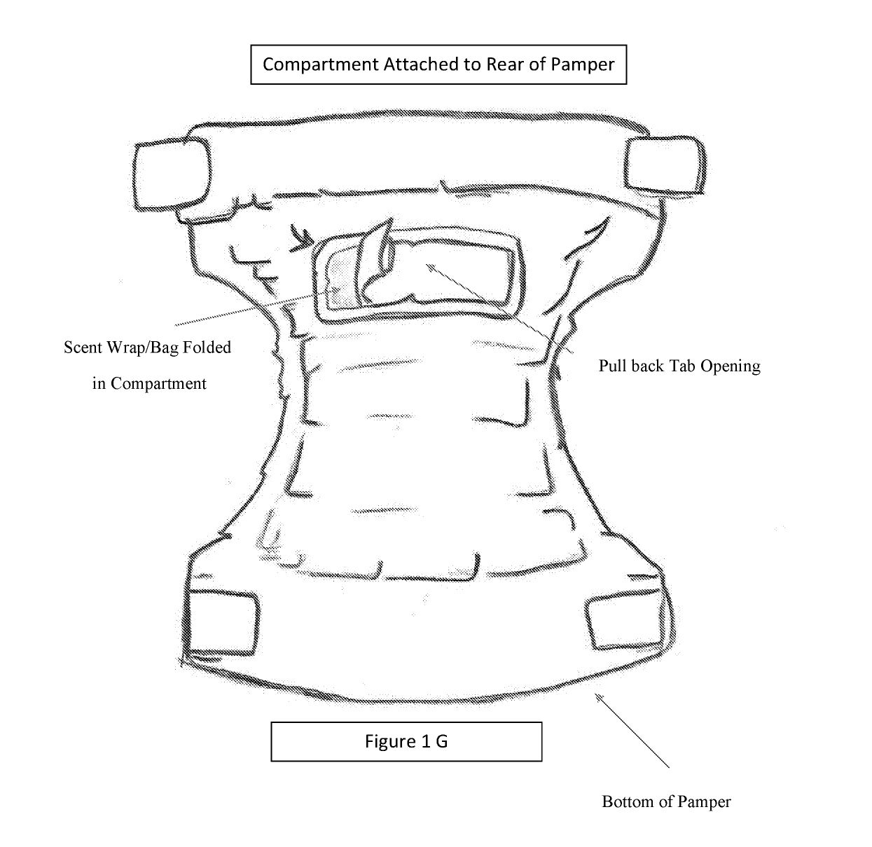

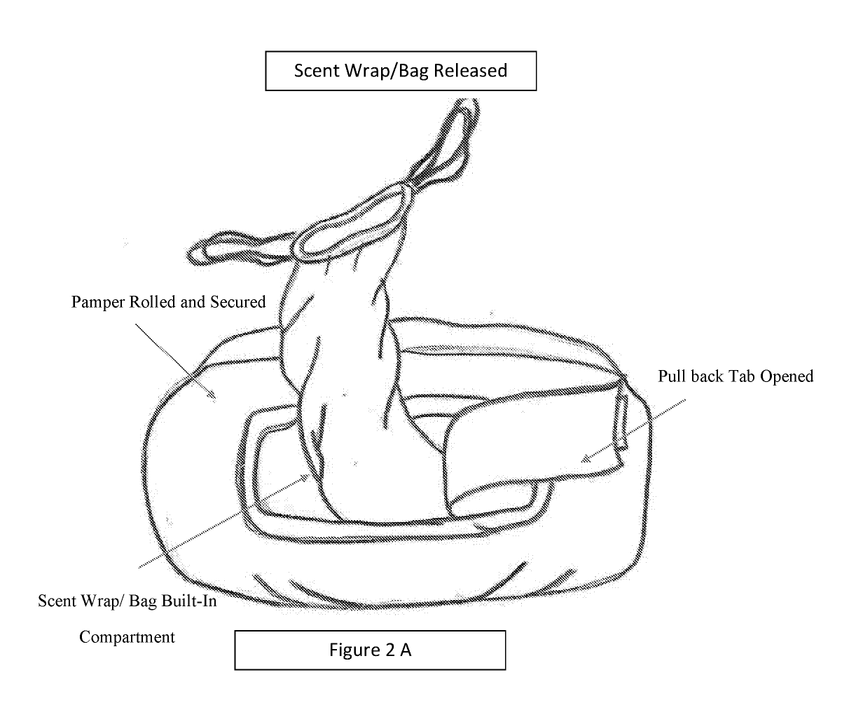

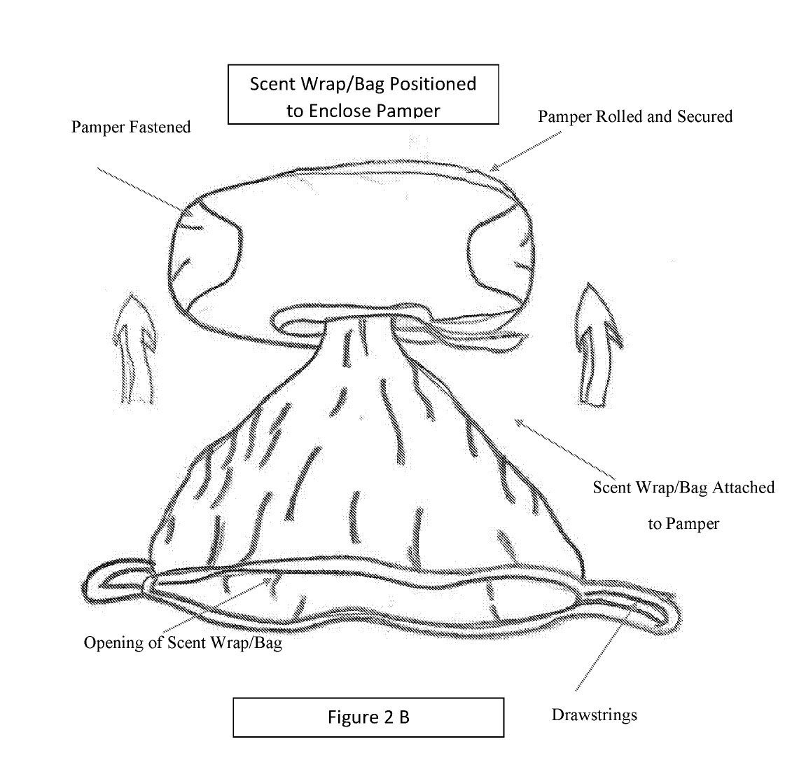

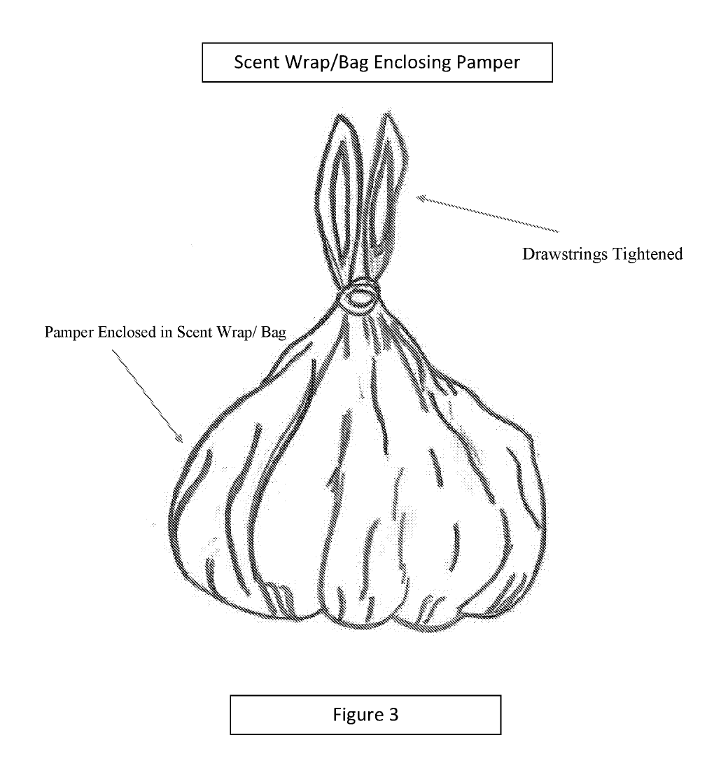

FIG. 1 A-E show the various positions of placing pull back tab on rear of pamper. FIG. 1F shows the preferred method of closed pull back tab placement on rear of pamper. FIG. 1G shows the pullback tab opening on the rear of a pamper/pull up. FIG. 2A a pamper/pull up is rolled and secured, pull tab on compartment to release built in disposable scent wrap/bag. FIG. 2B After pulling and releasing bag. Pull up and around pamper enclosing it attached built in scent wrap bag fully released. FIG. 3 Pamper is enclosed into wrap/bag, pull drawstring and tie.

### DETAILED DESCRIPTION OF THE INVENTION

FIG. 1A: A pamper/pull up compartment is a 4×1½ inch plastic section made of polyethylene. The compartment is fastened to the upper rear of a pamper/pull up and is non removable. The compartment materials, dimensions, perforations, locations all may vary depending on size of pamper manufacturing and safety requirements. The compartment includes a pullback tab on top in order to keep scented wrap/bag fresh and in place. The scent wrap bag is an 8 (H)×7.5 (L) inch single layer bag with thickness less than ½ A inch made of polyethylene terephthalate with draw strings. The wrap/bag has multiple scents and an odorless option for sensitive skin. The wrap bag is folded to maintain approximate dimension of 2 in. (H) by 2.35 (W) compressed and attached inside of the compartment. Plastic wrap/bag dimensions, scent, color material and enclosing apparatus may vary depending on size of pamper, manufacturing marketing and safety requirements. FIG. 2 A: When pullback tab is released the opening of the scent wrap/bag unfolds and the closed end of the wrap/bag remains attached to the compartment. FIG. 2B: At this point the used pamper is already rolled and sealed and ready to be closed in wrap/bag. Enclosed pamper by pulling the scent wrap/bag up and around a pamper/pull up and tighten with drawstrings as shown in FIG. 3.

### BRIEF SUMMARY OF THE INVENTION

The disposable scent wrap/bag is built into the upper rear of pamper. The scented bag is out of reach concealed and tucked away but easy access for use when needed. The plastic pullback tab allows the wrap/bag to remain in place concealing fresh scent until ready for use. This invention gives parents and caregivers an easier way to dispose of dirty pampers in a renewed and fresh process. Whether you are home or on the go the built in disposable wrap/bag is available to dispose of with freshness and convenience.

We claim:

1. A pamper/pull up comprising a disposable scent wrap, wherein the disposable scent wrap is located in the upper rear portion of a pamper/pull up.

2. A pamper/pull up of claim 1, wherein the disposable scent wrap is built into a pamper/pull up.

3. A pamper/pull up of claim 1, wherein the disposable scent wrap is fastened to said pamper.

4. A pamper/pull up of claim 3, wherein the disposable scent wrap is detachable.

5. A pamper/pull up of claim 1, wherein the disposable scent wrap is covered by a peel tab that conceals the disposable scent wrap.

6. A pamper/pull up of claim 1, wherein the disposable scent wrap has a draw string closure.

7. A pamper/pull up of claim 1, wherein said disposable scent wrap is sealed until opened for use.

8. A pamper/pull up of claim 1, wherein said disposable scent wrap has a perforated detachable pull tab.

9. A pamper/pull up of claim 1, wherein said wrap horizontally traverses the upper rear portion of a pamper/pull up.

10. A method of attaching a disposable scented wrap to a pamper comprising,
providing a pamper and attaching a disposable scented wrap.

11. The method of claim 10, wherein the wrap is attached to a pamper/pull up by way of a flat body of material that is opened by stick tab concealing the disposable scented wrap.

12. The method of claim 11, wherein the disposable scented wrap is detachable.

13. The method of claim 10, wherein the disposable scented wrap is attached to a pamper/pull up by way of a flat body of material that is opened by tear that conceals the disposable scented wrap.

14. The method of claim 13, wherein the disposable scented wrap is detachable.

15. The method of claim 10, where in the disposable scented wrap is attached by way of a separate detachable part that sticks to a pamper/pull up and conceals the disposable scented wrap.

16. A method of disposal for a pamper/pull up claim 1 comprising;
a. providing a plastic bag and inserting pamper into said plastic bag;
b. twisting said plastic bag so that it forms a neck portion to hold said contents from falling out of said plastic bag.

17. The method of claim 15, wherein said plastic bag comprises a drawstring to seal the bag.

18. The method of claim 15, wherein said plastic bag comprises a closure to seal the bag.

19. The method of claim 17, whereby said bag closure can be used to seal smell of pamper in said plastic bag.

20. The method of claim 11, wherein said flat body of material is composed of polyethylene terephthalate.

21. The method of claim 13, wherein said flat body of material is composed of polyethylene terephthalate.
